(19) United States
(12) Patent Application Publication
(10) Pub. No.: US 2005/0049693 A1
(43) Pub. Date: Mar. 3, 2005

(54) Medical devices and compositions for delivering biophosphonates to anatomical sites at risk for vascular disease

(75) Inventor: Jeffrey Walker, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular Inc., Santa Rosa, CA (US)

(21) Appl. No.: 10/924,562

(22) Filed: Aug. 24, 2004

Related U.S. Application Data
(60) Provisional application No. 60/497,772, filed on Aug. 25, 2003.

(51) Int. Cl. A61F 2/06

(52) U.S. Cl. 623/1.42

(57) ABSTRACT

Methods, compositions and devices for inhibiting restenosis are provided. Specifically, bisphosphonate compositions and medical devices useful for the site specific delivery of bisphosphonates are disclosed. In one embodiment the medical device is a vascular stent coated with a bisphosphonate selected from the group consisting of zolendronate and pamedronate and derivatives and analogues thereof. In another embodiment an injection catheter for delivery an anti-restenotic effective amount of bisphosphonate to the adventitia is provided.

### RELATED APPLICATIONS

This application claims priority to U.S. provisional patent application Ser. No. 60/497,772 filed Aug. 25, 2003.

### FIELD OF THE INVENTION

The present invention relates to medical devices and compositions for treating or preventing vascular disease. Specifically, the present invention relates the site specific delivery of anti-proliferative compounds using a medical device. More specifically, the present invention relates to devices for delivering bisphosphonates to regions of the mammalian vasculature at risk for restenosis.

### BACKGROUND OF THE INVENTION

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

Treating restenosis requires additional, generally more invasive, procedures including CABG in some cases. Consequently, methods for preventing restenosis, or treating incipient forms, are being aggressively pursued. One possible method for preventing restenosis is the administration of medicaments that block local invasion/activation of monocytes thus preventing the secretion of growth factors that may trigger VSMC proliferation and migration. Metabolic inhibitors such as anti-neoplastic agents are currently being investigated as potential anti-restenotic compounds. However, the toxicity associated with the systemic administration of metabolic inhibitors has recently stimulated research into in situ, site-specific drug delivery.

Anti-restenotic coated stents are one potential method of site-specific drug delivery. Once the coated stent is deployed, it releases the anti-restenotic agent directly into the tissue thus allowing for clinically effective drug concentrations to be achieved locally without subjecting the recipient to side effects associated with systemic drug delivery. Moreover, localized delivery of anti-proliferative drugs directly at the treatment site eliminates the need for specific cell targeting technologies.

Recently, significant research has been conducted utilizing compounds that inhibit cell cycle progression or completion. For convenience the mammalian cell cycle has been divided into four discrete segments. Mitosis and cell division occur in the M phase which lasts for only about one hour. This is followed by the G₁ phase (G for Gap) and then the S phase (S for syntheses) during which time DNA is replicated, and finally G₂ phase during which the cell prepares for mitosis. Eukaryotic cells in culture typically have cell cycle times of 16-24 hours; however, in some multicellular organisms the cell cycle can last for over 100 days. Furthermore, some cells such as neurons stop dividing completely in the mature mammal and are considered to be quiescent. This phase of the cell cycle is often referred to as G₀.

Variations in non-quiescence cell cycle times are largely dependent on the duration of the G₁ phase. Therefore, it is logical that a significant number of antiproliferative cell cycle inhibitors target cellular functions occurring during G₁. However, cell cycle inhibition is not limited to agents that selectively target the G₁ phase. For example, a number of cytotoxic compounds that either inhibit mitotic spindle formation or mitotic spindle separation are known. These compounds, such as paclitaxel target the M phase of the cell cycle. Compounds that affect DNA syntheses such as DNA topisomerases inhibitors block cell proliferation during the G₂ and S phase. However, regardless of the cell cycle phase affected, antiproliferative compounds target dividing cells and leave quiescent cells essentially undisturbed. This theory underlies the development of most anti-cancer chemotherapeutics.

Recently, bisphosphonates have been shown to possess anti-proliferative activity in addition to their previously recognized property of bone resporption inhibition. The bisphosphonate zoledronic acid (Zometa®) is of particular interest. Zoledronic acid is approved for treating bone metastases caused by prostrate, lung, renal, colorectal cancers and multiple myeloma. Bisphosphates, specifically Zometa®, are known to induce apoptosis and inhibit the angiogenic effects of bFGF (See for example Gschaidmeier, H, et al, 2000. Annals of Oncology, Vol 11 Suppl. 4 October 2000. for additional details). Successful cancer therapies based on zoledronic acid suggests that it may also be useful in treating other hyperproliferative diseases.

However, to date bisphosphonates have only been used systemically. Localized hyperproliferative diseases such as restenosis will most probably require site specific drug deployment using drug-releasing medical devices or direct drug injection. However, the effectiveness of localized therapies is highly variable and depends on balancing numerous synergistic and antagonistic physiological, mechanical and chemical factors. These factors include, but are not limited to, the size of the hyperproliferative lesion, the diffusability of the drug into tissue, the release kinetics obtained using various drug reservoir polymers. The solubility of the drug in these reservoir polymers and the overall inhibitory effect of the drug on the target cell. New anti-proliferative compounds may initially seem attractive candidates for treating restenosis; however, there is significant research, innovation and development involved before a successful new therapeutic modality is complete.

### SUMMARY OF THE INVENTION

The present invention relates to medical devices and methods for treating or inhibiting restenosis. Specifically, the present invention relates to devices for delivering bisphosponates to regions of the mammalian vasculature at risk for restenosis.

In one embodiment of the present invention a stent is adapted to deliver a bisphosphonate directly to the tissue of a mammalian lumen at risk for developing restenosis.

In another embodiment of the present invention the bisphosphonate is zoledronic acid or pamidronic acid.

In another embodiment of the present invention the stent adapted to deliver the bisphosphonate is a vascular stent and the mammalian anatomical lumen is a blood vessel.

In yet another embodiment of the present invention the vascular stent is delivered to the site at risk for restenosis within a blood vessel using a balloon catheter.

In another embodiment of the present innovation an injection catheter is used to deliver bisphosphonates to the adventitia at or near a site of restenosis, or an area susceptible to restenosis.

### BRIEF DESCRIPTION OF THE FIGURES

FIG. 1 depicts a vascular stent used to deliver the antirestenotic compounds of the present invention.

FIG. 2 depicts a balloon catheter assembly used for angioplasty and the site-specific delivery of stents to anatomical lumens at risk for restenosis.

FIG. 3 depicts the needle of an injection catheter in the retracted position (balloon deflated) according to the principles of the present invention where the shaft is mounted on an intravascular catheter.

FIGS. 4 and 5 illustrate use of the apparatus of FIG. 3 in delivering a substance into the adventitial tissue surrounding a blood vessel.

FIG. 6 graphically depicts the theoretical in vitro fast elution profile of zoledronic acid coated vascular stent.

FIG. 7 graphically depicts the theoretical in vitro slow elution profile of zoledronic acid coated vascular stent.

FIG. 8 graphically compares various theoretical in vitro elution profiles of zoledronic acid coated stents with theoretical in vivo elution profiles of zoledronic acid coated stents.

FIG. 9 graphically depicts the theoretical correlation between neointimal thickness and injury score in the combined proximal and distal stent segments in test pigs.

### DETAILED DESCRIPTION OF THE INVENTION

As previously discussed, bisphosphonates induce apoptosis and possess anti-angiogenic properties. Consequently, anti-proliferative bisphosphonates such as zoledronic acid and pamidronate may have significant potential as anti-restenostics. However, it should be understood that zoledronic acid and pamidronic acid are only exemplary embodiments of the present invention. Other suitable biphosphonates include those included in U.S. Pat. Nos. (USPNs) 4,777,163 and 4,939,130, (the entire contents of which are incorporated herein by reference) and others.

In one embodiment of the present invention the localized, or site-specific, delivery of an anti-restenotic composition comprising a compound having the general formula is provided: 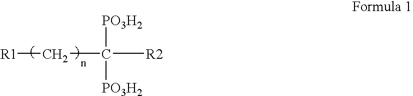
wherein R1 is an amine or is a substituted or unsubstituted heteroaromatic five-membered first ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C1-C4 alkyl, C1-C4 alkoxy, phenyl, cyclohexyl, cyclohexylmethyl, halogen and amino, and wherein two adjacent alkyl substitutents of R1 can together form a ring,
n is 0, 1 or 2

R2 is hydrogen, hydroxyl, amino, or an amino group substituted by C1-C4 alkyl, and as well as the pharmacologically acceptable salts and isomers thereof.

In yet another embodiment of the present invention bisphoponates of Formula 1 include but not limited to 1-hydroxy-2-imidazol-1yl phosphophonoethyl (zolendronate) or dihydrogen (3-amino-1-hydroxypropylidne) diphospate (pamedronate) are used as anti-restenotics.

The bisphosphonates of the present invention are delivered, alone or in combination with synergistic and/or additive therapeutic agents, directly to the affected area using medical devices. Potentially synergistic and/or additive therapeutic agents may include drugs that impact a different aspect of the restenosis process such as antiplatelet, antimigratory or antifibrotic agents. Alternately they may include drugs that also act as antiproliferatives and/or antiinflammatories but through a different mechanism than inhibiting molecular chaperone activity. For example, and not intended as a limitation, synergistic combinations considered within the scope of the present invention include at least one bisphosphonate and an antisense anti-c-myc oligonucleotide, at least one bisphosphonate and rapamycin or analogues and derivatives thereof such a 40-0-(2-hydroxyethyl)-rapamycin or tetrazole-containing rapamycin analogs (see, for example U.S. Pat. No. 6,015,815), at least one bisphosphonate and exochelin, at least one bisphosphonate and n-acetyl cysteine inhibitors, at least one bisphosphonate and a PPARγ agonist, and so on.

The medical devices used in accordance with the teachings of the present invention may be permanent medical implants, temporary implants, or removable devices. For examples, and not intended as a limitation, the medical devices of the present invention may include, stents, catheters, micro-particles, probes and vascular grafts.

In one embodiment of the present invention, stents are used as the drug delivery platform. The stents may be vascular stents, urethral stents, biliary stents, or stents intended for use in other ducts and organ lumens. Vascular stents may be used in peripheral, neurological or coronary applications. The stents may be rigid expandable stents or pliable self-expanding stents. Any biocompatible material may be used to fabricate the stents of the present invention including, without limitation, metals or polymers. The stents of the present invention may also be bioresorbable.

In one embodiment of the present invention, vascular stents are implanted into coronary arteries immediately following angioplasty. However, one significant problem associated with stent implantation, specifically vascular stent deployment, is restenosis. Restenosis is a process whereby a previously opened lumen is re-occluded by VSMC proliferation. Therefore, it is an object of the present invention to provide stents that suppress or eliminate VSMC migration and proliferation and thereby reduce, and/or prevent restenosis.

Vascular stents and stent grafts (referred to hereinafter collectively as “stents”) are used to relieve the symptoms associated with coronary artery disease caused by occlusion in one or more coronary artery or aneurysms. Occluded coronary arteries result in diminished blood flow to heart muscles causing ischemia induced angina and in severe cases myocardial infarcts and death. Stents are generally deployed using catheters having the stent attached to an inflatable balloon at the catheter's distal end. The catheter is inserted into an artery and guided to the deployment site. In many cases the catheter is inserted into the femoral artery or of the leg or carotid artery and the stent is deployed deep within the coronary vasculature at an occlusion site.

Vulnerable plaque stabilization is another application for coated drug-eluting vascular stents. Vulnerable plaque is composed of a thin fibrous cap covering a liquid-like core composed of an atheromatous gruel. The exact composition of mature atherosclerotic plaques varies considerably and the factors that effect an atherosclerotic plaque's make-up are poorly understood. However, the fibrous cap associated with many atherosclerotic plaques is formed from a connective tissue matrix of smooth muscle cells, types I and III collagen and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. (G. Pasterkamp and E. Falk. 2000. Atherosclerotic Plaque Rupture: An Overview. J. Clin. Basic Cardiol. 3:81-86). The ratio of fibrous cap material to atheromatous gruel determines plaque stability and type. When atherosclerotic plaque is prone to rupture due to instability it is referred to a “vulnerable” plaque. Upon rupture the atheromatous gruel is released into the blood stream and induces a massive thrombogenic response leading to sudden coronary death. Recently, it has been postulated that vulnerable plaque can be stabilized by stenting the plaque. Moreover, vascular stents having a drug-releasing coating composed of matrix metalloproteinase inhibitor (such as, but not limited to, tetracycline-class antibiotics) dispersed in, or coated with (or both) a polymer may further stabilize the plaque and eventually lead to complete healing.

Treatment of aneurysms is another application for drug-eluting stents. An aneurysm is a bulging or ballooning of a blood vessel usually caused by atherosclerosis, aneurysms occur most often in the abdominal portion of the aorta. At least 15,000 Americans die each year from ruptured abdominal aneurysms. Back and abdominal pain, both symptoms of an abdominal aortic aneurysm, often do not appear until the aneurysm is about to rupture, a condition that is usually fatal. Stent grafting has recently emerged as an alternative to the standard invasive surgery. A vascular graft containing a stent (stent graft) is placed within the artery at the site of the aneurysm and acts as a barrier between the blood and the weakened wall of the artery, thereby decreasing the pressure on artery. The less invasive approach of stent-grafting aneurysms decreases the morbidity seen with conventional aneurysm repair. Additionally, patients whose multiple medical comorbidities make them excessively high risk for conventional aneurysm repair are candidates for stent-grafting. Stent grafting has also emerged as a new treatment for a related condition, acute blunt aortic injury, where trauma causes damage to the artery.

Once positioned at the treatment site, the stent or graft is deployed. Generally, stents are deployed using balloon catheters. The balloon expands the sent gently compressing it against the arterial lumen clearing the vascular occlusion or stabilizing the plaque. The catheter is then removed and the stent remains in place permanently. Most patients return to a normal life following a suitable recovery period and have no reoccurrence of the arterial disease associated with the stented deployment. However, in some cases the arterial wall's initma is damaged either by the disease process itself or as the result of stent deployment. This injury initiates a complex biological response culminating in vascular smooth muscle cell hyperproliferation and occlusion, or restenosis, at the stent site.

Recently significant efforts have been devoted to preventing restenosis. Several techniques including brachytherapy, excimer laser, and pharmacological interventions have been developed. The least invasive and most promising treatment modality is the pharmacological approach. A preferred pharmacological approach involves the site specific delivery of cytostatic or cytotoxic drugs directly to the stent deployment area. Site specific delivery is preferred over systemic delivery for several reasons. First, many cytostatic and cytotoxic drugs are highly toxic and cannot be administered systemically at concentrations needed to prevent restenosis. Moreover, the systemic administration of drugs can have unintended side effects at body locations remote from the treatment site. Additionally, many drugs are either not sufficiently soluble, or too quickly cleared from the blood stream to effectively prevent restenosis. Therefore, administration of anti-restenotic compounds directly to the treatment area is preferred.

In one embodiment of the present invention metallic vascular stents are coated with one or more anti-restenotic compound, specifically at least one bisphosphonate, more specifically the bisphosphonate is zolendronate. The bisphosphonate may be dissolved or suspended in any carrier compound that provides a stable composition that does not react adversely with the device to be coated or inactivate the bisphosphonate. The metallic stent is provided with a biologically active bisphosphonate coating using any technique known to those skilled in the art of medical device manufacturing. Suitable non-limiting examples include impregnation, spraying, brushing, dipping and rolling. After the bisphosphonate solution is applied to the stent it is dried leaving behind a stable bisphosphonate delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the bisphosphonate compositions of the present invention.

The anti-restenotic effective amounts of bisphosphonates used in accordance with the teachings of the present invention can be determined by a titration process. Titration is accomplished by preparing a series of stent sets. Each stent set will be coated with, or contain, different dosages of the bisphosphonate selected. The highest concentration used will be partially based on the known toxicology of the compound. The maximum amount of drug delivered by the stents made in accordance with the teaching of the present invention will fall below known toxic levels. Each stent set will be tested in vivo using the preferred animal model as described in Example 5 below. The dosage selected for further studies will be the minimum dose required to achieve the desired clinical outcome. In the case of the present invention, the desired clinical outcome is defined as the inhibition of vascular re-occlusion, or restenosis. Generally, and not intended as a limitation, an anti-restenotic effective amount of the bisphosphonates of the present invention will range between about 0.5 ng to 1.0 mg depending on the particular bisphosphonate used and the delivery platform selected.

In addition to the bisphosphonate selected, treatment efficacy may also be affected by factors including dosage, route of delivery and the extent of the disease process (treatment area). An effective amount of a bisphosphonate composition can be ascertained using methods known to those having ordinary skill in the art of medicinal chemistry and pharmacology. First the toxicological profile for a given bisphosphonate composition is established using standard laboratory methods. For example, the candidate bisphosphonate composition is tested at various concentration in vitro using cell culture systems in order to determine cytotoxicity. Once a non-toxic, or minimally toxic, concentration range is established, the bisphosphonate composition is tested throughout that range in vivo using a suitable animal model. After establishing the in vitro and in vivo toxicological profile for the bisphosphonate compound, it is tested in vitro to ascertain if the compound retains antiproliferative activity at the non-toxic, or minimally toxic ranges established.

Finally, the candidate bisphosphonate composition is administered to treatment areas in humans in accordance with either approved Food and Drug Administration (FDA) clinical trial protocols, or protocol approved by Institutional Review Boards (IRB) having authority to recommend and approve human clinical trials for minimally invasive procedures. Treatment areas are selected using angiographic techniques or other suitable methods known to those having ordinary skill in the art of intervention cardiology. The candidate bisphosphonate composition is then applied to the selected treatment areas using a range of doses. Preferably, the optimum dosages will be the highest non-toxic, or minimally toxic concentration established for the bisphosphonate composition being tested. Clinical follow-up will be conducted as required to monitor treatment efficacy and in vivo toxicity. Such intervals will be determined based on the clinical experience of the skilled practitioner and/or those established in the clinical trial protocols in collaboration with the investigator and the FDA or IRB supervising the study.

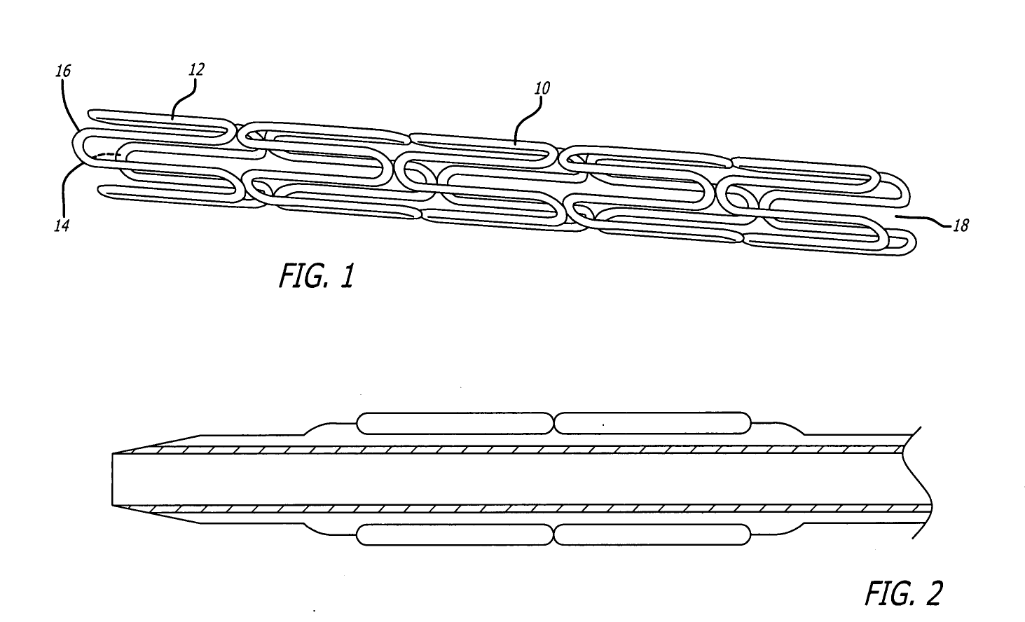

The bisphosphonate therapy of the present invention can be administered directly to the treatment area using any number of techniques and/or medical devices. In one embodiment of the present invention the bisphosphonate composition is applied to a vascular stent. The vascular stent can be of any composition or design. For example, the stent may be self-expanding or mechanically expanded stent 10 (FIG. 1) using a balloon catheter (FIG. 2). The stent 10 may be made from stainless steel, titanium alloys, nickel alloys or biocompatible polymers. Furthermore, the stent 10 may be polymeric or a metallic stent coated with at least one polymer. In other embodiments the delivery device is an aneurysm shield, a vascular graft or surgical patch. In yet other embodiments the bisphosphonate therapy of the present invention is delivered using a porous or “weeping” catheter to deliver a bisphosphonate containing hydrogel composition to the treatment area. Still other embodiments include microparticles delivered using a catheter or other intravascular or transmyocardial device.

In another embodiment an injection catheter can be used to deliver the bisphosphonates of the present invention either directly into, or adjacent to, a vascular occlusion or a vasculature site at risk for developing restenosis (treatment area). As used herein, adjacent means a point in the vasculature either distal to, or proximal from a treatment area that is sufficiently close enough for the anti-restenotic composition to reach the treatment area at therapeutic levels. A vascular site at risk for developing restenosis is defined as a treatment area where a procedure is conducted that may potentially damage the luminal lining. Non-limiting examples of procedures that increase the risk of developing restenosis include angioplasty, stent deployment, vascular grafts, ablation therapy, and brachytherapy.

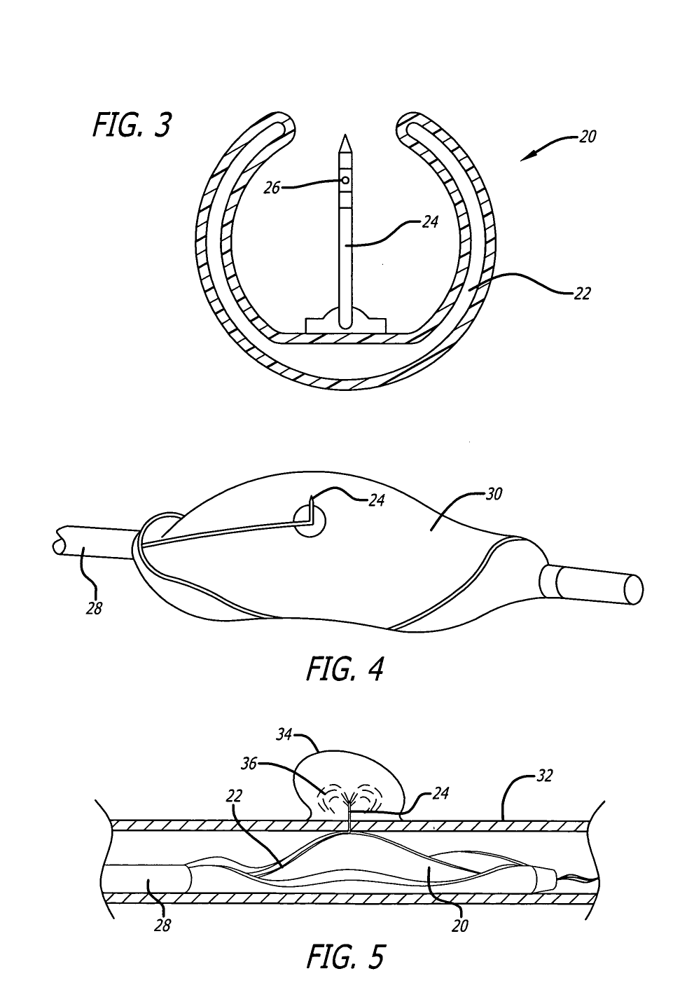

In one embodiment of the present invention an injection catheter as depicted in U.S. Pat. No. 6,547,803, and pending patent application No. 09/961,079 can be used to administer the bisphosphonates of the present invention directly to the adventia. FIGS. 3, 4 and 5 depict one such embodiment. FIG. 3 illustrates the C-shaped configuration of the catheter balloon 20 prior to inflation having the injection needle 24 nested therein and a balloon interior 22 connected to an inflation source (not shown) which permits the catheter body to be expanded as shown in FIG. 4. Needle 24 has an injection port 26 that transits the bisphosphonate into the adventia from a proximal reservoir (not shown) located outside the patient.

FIG. 4 illustrates the inflated balloon 30 attached to the catheter body 28 and injection needle 24 capable of penetrating the adventia. FIG. 5 depicts deployment of the bisphosphonate of the present invention directly into the adventia 34. The injection needle 24 penetrates the blood vessel wall 32 as balloon 20 is inflated and injects the bisphosphonate 36 into the tissue.

The medical device can be made of virtually any biocompatible material having physical properties suitable for the design. For example, tantalum, stainless steel and nitinol have been proven suitable for many medical devices and could be used in the present invention. Also, medical devices made with biostable or bioabsorbable polymers can be used in accordance with the teachings of the present invention. Although the medical device surface should be clean and free from contaminants that may be introduced during manufacturing, the medical device surface requires no particular surface treatment in order to retain the coating applied in the present invention. Both surfaces (inner 14 and outer 12 of stent 10, or top and bottom depending on the medical devices' configuration) of the medical device may be provided with the coating according to the present invention.

In order to provide the coated medical device according to the present invention, a solution which includes a solvent, a polymer dissolved in the solvent and a bisphosphonate composition dispersed in the solvent is first prepared. It is important to choose a solvent, a polymer and a therapeutic substance that are mutually compatible. It is essential that the solvent is capable of placing the polymer into solution at the concentration desired in the solution. It is also essential that the solvent and polymer chosen do not chemically alter the bisphosphonate's therapeutic character. However, the bisphosphonate composition only needs to be dispersed throughout the solvent so that it may be either in a true solution with the solvent or dispersed in fine particles in the solvent. The solution is applied to the medical device and the solvent is allowed to evaporate leaving a coating on the medical device comprising the polymer(s) and the bisphosphonate composition.

Typically, the solution can be applied to the medical device by either spraying the solution onto the medical device or immersing the medical device in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the medical device. In either a coating applied by spraying or by immersion, multiple application steps are generally desirable to provide improved coating uniformity and improved control over the amount of bisphosphonate composition to be applied to the medical device. The total thickness of the polymeric coating will range from approximately 1 micron to about 20 microns or greater. In one embodiment of the present invention the bisphosphonate composition is contained within a base coat, and a top coat is applied over the bisphosphonate containing base coat to control release of the bisphosphonate into the tissue.

The polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer-to-bisphosphonate composition ratio will depend on the efficacy of the polymer in securing the bisphosphonate composition onto the medical device and the rate at which the coating is to release the bisphosphonate composition to the tissue of the blood vessel. More polymer may be needed if it has relatively poor efficacy in retaining the bisphosphonate composition on the medical device and more polymer may be needed in order to provide an elution matrix that limits the elution of a very soluble bisphosphonate composition. A wide ratio of therapeutic substance-to-polymer could therefore be appropriate and could range from about 0.1% to 99% by weight of therapeutic substance-to-polymer.

In one embodiment of the present invention a vascular stent as depicted in FIG.1 is coated with bisphosphonates using a two-layer biologically stable polymeric matrix comprised of a base layer and an outer layer. Stent 10 has a generally cylindrical shape and an outer surface 12, an inner surface 14, a first open end 16, a second open end 18 and wherein the outer and inner surfaces 12, 14 are adapted to deliver an anti-restenotic effective amount of at least one bisphosphonate in accordance with the teachings of the present invention. Briefly, a polymer base layer comprising a solution of ethylene-co-vinylacetate and polybutylmethacrylate is applied to stent 10 such that the outer surface 12 is coated with polymer. In another embodiment both the inner surface 14 and outer surface 12 of stent 10 are provided with polymer base layers. The bisphosphonate or mixture thereof is incorporated into the base layer. Next, an outer layer comprising only polybutylmethacrylate is applied to stent's 10 outer layer 14 that has been previous provide with a base layer. In another embodiment both the inner surface 14 and outer surface 12 of stent 10 are proved with polymer outer layers.

The thickness of the polybutylmethacrylate outer layer determines the rate at which the bisphosphonates elute from the base coat by acting as a diffusion barrier. The ethylene-co-vinylacetate, polybutylmethacrylate and bisphosphonate solution may be incorporated into or onto a medical device in a number of ways. In one embodiment of the present invention the bisphosphonate/polymer solution is sprayed onto the stent 10 and then allowed to dry. In another embodiment, the solution may be electrically charged to one polarity and the stent 10 electrically changed to the opposite polarity. In this manner, the bisphosphonate/polymer solution and stent will be attracted to one another thus reducing waste and providing more control over the coating thickness.

In another embodiment of the present invention the bisphosphonate is any bisphosphonate and the polymer is bioresorbable. The bioresorbable polymer-bisphosphonate blends of the present invention can be designed such that the polymer absorption rate controls drug release. In one embodiment of the present invention a polycaprolactone-zoledronic acid blend is prepared. A stent 10 is then stably coated with the polycaprolactone-zoledronic acid blend wherein the stent coating has a thickness of between approximately 0.1 μm to approximately 100 μm. The polymer coating thickness determines the total amount of zoledronic acid delivered and the polymer's absorption rate determines the administrate rate.

Using the preceding examples it is possible for one of ordinary skill in the part of polymer chemistry to design coatings having a wide range of dosages and administration rates. Furthermore, drug delivery rates and concentrations can also be controlled using non-polymer containing coatings and techniques known to persons skilled in the art of medicinal chemistry and medical device manufacturing,

The following examples are provided to more precisely define and enable the bisphosphonate-eluting medical devices of the present invention. It is understood that there are numerous other embodiments and methods of using the present invention that will be apparent embodiments to those of ordinary skill in the art after having read and understood this specification and examples. Moreover, it is understood that bisphosphonates, specifically zoledronic acid, is but one example of the bisphosphonates that can be used according to the teachings of the present invention. These alternate embodiments are considered part of the present invention.

In the following Examples two boiocompatibe polymers, polycaprolactone and polyvinyl pyrrolidone (PVP) have been used as exemplary embodiment. However, it is understood that other embodiments include other monomers such as acrylates, urethanes, cyanates, peroxides, styrenes and many others. Copolymers including bipolymes and terpolymers may also be used. Copolymers may be block copolymers, random or segmented homochain copolymers. The polymers may have pendent groups and may or may not be cross-linked. The optimum polymer-bisphosphonate composition will ultimately be determined using the drug and polymer relative solubility constants, the physical, biological and drug-release kinetics desired for a specific application. For more detail please see U.S. patent application Ser. No. 60/495,143 incorporated herein by reference (Attorney docket number 51288-00039/P1366).

### EXAMPLE 1

### Metal Stent Cleaning Procedure

Stainless steel stents are placed a glass beaker and covered with reagent grade or better hexane. The beaker containing the hexane immersed stents is then placed into an ultrasonic water bath and treated for 15 minutes at a frequency of between approximately 25 to 50 KHz. Next the stents are removed from the hexane and the hexane is discarded. The stents are then immersed in reagent grade or better 2-propanol and vessel containing the stents and the 2-propanol is treated in an ultrasonic water bath as before. Following cleaning the stents with organic solvents, they are thoroughly washed with distilled water and thereafter immersed in 1.0 N sodium hydroxide solution and treated at in an ultrasonic water bath as before. Finally, the stents are removed from the sodium hydroxide, thoroughly rinsed in distilled water and dried in a vacuum oven overnight at 40° C.

After cooling the dried stents to room temperature in a desiccated environment they are weighed and their weights are recorded.

### EXAMPLE 2

### Coating a Clean, Dried Stent Using a Drug/Polymer System

Zoledronic acid (250 μg) is carefully weighed and added to a small neck glass bottle containing 27.56 mL of tetrahydofuran (THF). The zoledronic acid-THF suspension is then thoroughly mixed until a clear solution is achieved.

Next 251.6 mg of polycaprolactone (PCL) is added to the zoledronic acid-THF solution and mixed until the PCL dissolved forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. overnight. The dried, coated stents are weighed and the weights recorded.

The concentration of drug loaded onto the stents is determined based on the final coating weight. Final coating weight is calculated by subtracting the stent's pre-coating weight from the weight of the dried, coated stent.

### EXAMPLE 3

### Coating a Clean, Dried Stent Using a Sandwich-type Coating

In one embodiment of the present invention a cleaned, dry stent is first coated with PVP or another suitable polymer followed by a coating of zoledronic acid. Finally, a second coating of PVP is provided to seal the stent thus creating a PVP-zoledronic acid-PVP sandwich coated stent. In another embodiment a parylene primer is applied to the bare metal stent prior to applying the zoledronic acid-containing polymer coating. In yet another embodiment, a polymer cap coat is applied over the zoledronic acid coating wherein the cap coat comprises a different polymer from the polymer used in the zoledronic acid-containing polymer coating.

In another embodiment of the present invention a polybutylmethacrylate-polyethylene vinyl acetate polymer blend is used to control the release of zoledronic acid.

The following example is not intended as a limitation but only as one possible polymer coating that can be used in accordance with the teachings of the present invention. Other coatings will be discussed herein and are considered within the scope of the present invention.

The Sandwich Coating Procedure: 100 mg of PVP is added to a 50 mL Erlenmeyer containing 12.5 mL of THF. The flask is carefully mixed until all of the PVP is dissolved. In a separate clean, dry Erlenmeyer flask 250 μg of zoledronic acid is added to 11 mL of THF and mixed until dissolved.

A clean, dried stent is then sprayed with PVP until a smooth confluent polymer layer is achieved. The stent is then dried in a vacuum oven at 50° C. for 30 minutes.

Next nine successive layers of the zoledronic acid are applied to the polymer-coated stent. The stent is allowed to dry between each of the successive zoledronic acid coats. After the final zoledronic acid coating has dried, three successive coats of PVP are applied to the stent followed by drying the coated stent in a vacuum oven at 50° C. over night. The dried, coated stent is weighed and its weight recorded.

The concentration of drug in the drug/polymer solution and the final amount of drug loaded onto the stent determine the final coating weight. Final coating weight is calculated by subtracting the stent's pre-coating weight from the weight of the dried, coated stent.

### EXAMPLE 4

### Coating a Clean, Dried Stent with Pure Drug

One microgram of zoledronic acid is carefully weighed and added to a small neck glass bottle containing 11.4 mL of absolute methanol (MeOH). The zoledronic acid-methanol suspension is then heated at 50° C. for 15 minutes and then mixed until the zoledronic acid is completely dissolved.

Next a clean, dried stent is mounted over the balloon portion of angioplasty balloon catheter assembly. The stent is then sprayed with, or in an alternative embodiment, dipped into, the zoledronic acid-MeOH solution. The coated stent is dried in a vacuum oven at 50° C. overnight. The dried, coated stent is weighed and its weight recorded.

The concentration of drug loaded onto the stents is determined based on the final coating weight. Final coating weight is calculated by subtracting the stent's pre-coating weight from the weight of the dried, coated stent.

### EXAMPLE 5

### In vivo Testinq of a Bisphosphonate-coated Vascular Stent in a Porcine Model

The ability of a bisphosphonates to reduce neointimal hyperplasia in response to intravascular stent placement in an acutely injured porcine coronary artery is demonstrated in the following example. Two controls and three treatment arms are used as outlined below:

Control Groups: Six animals are used in each control group. The first control group tests the anti-restenotic effects of the clean, dried Medtronic Vascular S7 stents having neither polymer nor drug coatings. The second control group tests the anti-restenotic effects of polymer alone. Clean, dried Medtronic Vascular S7 stents having polybutylmethacrylate-polyethylene vinyl acetate polymer blend coatings without drug are used in the second control group.

Experimental Treatment Groups: Twelve animals are included in each group.
Experimental group 1 is treated with Medtronic Vascular S7 stents having a coating comprised of a 75:25 polybutylmethacrylate-polyethylene vinyl acetate polymer blend containing 10% zoledronic acid by weight are designated the fast release group in accordance with the teachings of the present invention.
Experimental group 2 is treated with Medtronic Vascular S7 stents having a coating comprised of a 80:20 polybutylmethacrylate-polyethylene vinyl acetate polymer blend containing 10% zoledronic acid by weight are designated the slow release group in accordance with the teachings of the present invention.

The swine has emerged as the most appropriate animal model for the study of the endovascular devices. The anatomy and size of the coronary vessels are comparable to that of humans. Furthermore, the neointimal hyperplasia that occurs in response to vascular injury is similar to that seen clinically in humans. Results obtained in the swine animal model are considered predictive of clinical outcomes in humans. Consequently, regulatory agencies have deemed six-month data in the porcine sufficient to allow progression to human trials. Therefore, as used herein “animal” shall include mammals, fish, reptiles and birds. Mammals include, but are not limited to, primates, including humans, dogs, cats, goats, sheep, rabbits, pigs, horses and cows.

Non-atherosclerotic acutely injured right carotid artery (RCA), left anterior descending (LAD), and/or left circumflex (LCX) arteries of the Farm Swine (or miniswine) are utilized in this study. Placement of coated and control stents is random by animal and by artery. The animals are handled and maintained in accordance with the requirements of the Laboratory Animal Welfare Act (P.L.89-544) and its 1970 (P.L. 91-579), 1976 (P.L. 94-279), and 1985 (P.L. 99-198) amendments. Compliance is accomplished by conforming to the standards in the Guide for the Care and the Use of Laboratory Animals, ILAR, National Academy Press, revised 1996. A veterinarian performs a physical examination on each animal during the pre-test period to ensure that only healthy pigs are used in this study.

Pre-Operative Procedures: The animals are monitored and observed 3 to 5 days prior to experimental use. The animals had their weight estimated at least 3 days prior to the procedure in order to provide appropriate drug dose adjustments for body weight. At least one day before stent placement, 650 mg of aspirin is administered. Animals are fasted twelve hours prior to the procedure.

Anesthesia: Anesthesia is induced in the animal using intramuscular (I.M.) Telazol and Xylazine. Atropine is administered (20 μg/kg I.M.) to control respiratory and salivary secretions. Upon induction of light anesthesia, the subject animal is intubated. Isoflurane (0.1 to 5.0% to effect by inhalation) in oxygen is administered to maintain a surgical plane of anesthesia. Continuous electrocardiographic monitoring is performed. An intravenous (I.V.) catheter is placed in the ear vein in case it is necessary to replace lost blood volume. The level of anesthesia is monitored continuously by electrocardiogram (ECG) and the animal's response to stimuli.

Catheterization and Stent Placement: Following induction of anesthesia, the surgical access site is shaved and scrubbed with chlorohexidine soap. An incision is made in the region of the right or left femoral (or carotid) artery and betadine solution is applied to the surgical site. An arterial sheath is introduced via an arterial stick or cutdown and the sheath is advanced into the artery. A guiding-catheter is placed into the sheath and advanced via a 0.035″ guide wire as needed under fluoroscopic guidance into the ostium of the coronary arteries. An arterial blood sample is obtained for baseline blood gas, ACT and HCT. Heparin (200 units/kg) is administered as needed to achieve and maintain ACT ≧300 seconds. Arterial blood pressure, heart rate, and ECG are recorded.

After placement of the guide catheter into the ostium of the appropriate coronary artery, angiographic images of the vessels are obtained in at least two orthagonal views to identify the proper location for the deployment site. Quantitative coronary angiography (QCA) is performed and recorded. Nitroglycerin (200 μg I.C.) [intracardial] may be administered prior to treatment and as needed to control arterial vasospasm. The delivery system is prepped by aspirating the balloon with negative pressure for five seconds and by flushing the guidewire lumen with heparinized saline solution.

Deployment, patency and positioning of stent are assessed by angiography and a TIMI score is recorded. The Thrombolysis In Myocardial Infarction (TIMI) risk score predicts adverse clinical outcomes in patients with non-ST-elevation acute coronary syndromes (NSTEACS). cardiac catheterization, the TIMI risk score correlated with the extent and severity of CAD. Results are recorded on video and cine. Final lumen dimensions are measured with QCA and/or intravascular ultrasound (IVUS). These procedures are repeated until a device is implanted in each of the three major coronary arteries of the pig. The stents are deployed having an expansion ratio of 1:1.2. After final implant, the animal is allowed to recover from anesthesia. Aspirin is administered at 325 mg orally daily until sacrificed 28 days later.

Follow-up Procedures and Termination: After 28 days, the animals are anesthetized and a 6 F arterial sheath is introduced and advanced. A 6 F large lumen guiding-catheter (diagnostic guide) is placed into the sheath and advanced over a guide wire under fluoroscopic guidance into the coronary arteries. After placement of the guide catheter into the appropriate coronary ostium, angiographic images of the vessel are taken to evaluate the stented sites. At the end of the re-look procedure, the animals are euthanized with an overdose of pentabarbitol I.V. and KCI I.V. The heart, kidneys, and liver are harvested and visually examined for any external or internal trauma. The organs are flushed with 1000 mL of lactated ringers at 100 mmHg and then flushed with 1000 mL of formalin at 100-120 mmHg. All organs are stored in labeled containers of formalin solution.

Histology and Pathology: The stented vessels are X-rayed prior to histology processing. The stented segments are processed for routine histology, sectioned, and stained following standard histology lab protocols. Appropriate stains are applied in alternate fashion on serial sections through the length of the treated vessels.

Data Analysis and Statistics

QCA Measurement: Quantitative angiography is performed to measure the balloon size at peak inflation as well as vessel diameter pre- and post-stent placement and at the 28 day follow-up. The following data are measured or calculated from angiographic data:
Stent-to-artery-ratio
Minimum lumen diameter (MLD)
Distal and proximal reference lumen diameter
Percent Stenosis =(Minimum lumen diameter ÷reference lumen diameter)×100

Histomorphometric analysis: Histologic measurements are made from sections from the native proximal and distal vessel and proximal, middle, and distal portions of the stent. A vessel injury score is calculated using the method described by Schwartz et al. (Schwartz RS et al. Restenosis and the proportional neointimal response to coronary artery injury: results in a porcine model. J Am Coll Cardiol 1992; 19:267-74). The mean injury score for each arterial segment is calculated. Investigators scoring arterial segment and performing histopathology are “blinded” to the device type. The following measurements are determined:
External elastic lamina (EEL) area
Internal elastic lamina (IEL) area
Luminal area
Adventitial area
Mean neointimal thickness
Mean injury score

The neointimal area and the % of in-stent restenosis are calculated as follows:
Neointimal area=(IEL−luminal area)
In-stent restenosis=[1−(luminal area÷IEL)]×100.

A given treatment arm is deemed beneficial if treatment results in a significant reduction in neointimal area and/or in-stent restenosis compared to both the bare stent control and the polymer-on control.

The following surgical supplies and equipment are required for the procedures described above:
1. Standard vascular access surgical tray
2. Non-ionic contrast solution
3. ACT machine and accessories
4. HCT machine and accessories (if applicable)
5. Respiratory and hemodynamic monitoring system
6. IPPB Ventilator, associated breathing circuits and Gas Anesthesia Machine
7. Blood gas analysis equipment
8. 0.035″ HTF or Wholey modified J guidewire, 0.014″ Guidewires
9. 6, 7, 8, and 9 F introducer sheaths and guiding catheters (as applicable)
10. Cineangiography equipment with QCA capabilities
11. Ambulatory defibrillator
12. Standard angioplasty equipment and accessories
13. IVUS equipment (if applicable)
14. For radioactive labeled cell studies (if applicable):
15. Centrifuge
16. Aggregometer
17. Indium 111 oxime or other as specified
18. Automated Platelet Counter
19. Radiation Detection Device

Results: The results of the animal experiments are depicted in FIG. 9. FIG. 9 graphically depicts 28-day efficacy studies in farm swine. Medtronic S7 stents (18 mm ×3-3.5 mm diameter) are coated as described herein are sterilized and implanted into farm swine at an expansion ratio of 1:1.2 as described above. Animals are allowed to recover, and held for 28 d, after which the animal is euthanized and the tissue fixed and processed for histochemistry and histomorphometry, using standard techniques. FIG. 9 graphically depicts the correlation between neointimal thickness and injury score in the combined proximal and distal stent segments. The neointimal thickness and injury score are measured at each strut of the stent. A good correlation is observed between the injury score and neointimal thickness in the bare stent control group. A significant decrease in the neointimal thickness when the injury score increases is observed when the data from the “fast-release” stent is compared with the “slow-release” and bare stent controls. In FIG. 9 solid diamonds depict the bare metal Medtronic Vascular S7 control stent; squares depict Medtronic Vascular S7 control stents having a polymer-only coating (no drug); triangles depict Medtronic Vascular S7 stents having the “fast elution profile” coatings and diamonds depict Medtronic Vascular S7 stents having the “slow elution profile” coatings. These results clearly demonstrate the fast release zoledronic acid containing coatings provide stents having reduced mean injury scores when compared to the controls.

### EXAMPLE 6

### Inhibition of Human Coronary Artery Smooth Muscle Cells by Zoledronic Acid

Materials
1. Human coronary smooth muscles cells (HCASMC) are obtained from Clonetics, a division of Cambrex, Inc.
2. HCASMC basal media, supplied by Clonetics and supplemented with fetal bovine serum, insulin, hFGF-B (human fibroblast growth factor) hEGF (human epidermal growth factor).
3. Zoledronic acid, (Availbale through Novartis Pharmaceuticals, Inc.)
4. Absolute methanol
5. Twenty-four well polystyrene tissue culture plates

Human coronary artery smooth muscle cells proliferation inhibition studies: Human coronary smooth muscles cells (HCASMC) are seeded in 24 well polystyrene tissue culture plates at a density of 5×10³ cells per well. Two different feeding and reading strategies are employed.

Strategy 1: Cells are plated in cell culture media containing various concentrations of zoledronic acid (see Table 1) and incubated at 37° C. for 48 hours. After the initial 48 hour incubation, the zoledronic acid containing plating media is changed and the cells are fed with drug free media, incubated for an additional 48 hours and then read.

Strategy 2: Cells are plated in cell culture media containing various concentrations of zoledronic acid (see Table 1) and incubated at 37° C. for 48 hours. After the initial 48 hours incubation, the zoledronic acid-containing plating media is changed and the cells are fed with zoledronic acid-containing media and incubated for an additional 48 hours and then read.

A 0.5 mg/mL stock solution of zoledronic acid is prepared in absolute methanol and diluted to the following final test concentrations in cell culture media:

TABLE 1
Test Concentrations of zoledronic acid used in vitro.

| nM zoledronic acid | ng/ml zoledronic acid |
|---|---|
| 0 | 0 |
| 0.1 | 0.06 |
| 0.5 | 0.28 |
| 1 | 0.56 |
| 5 | 2.8 |
| 10 | 5.61 |
| 50 | 28.03 |
| 100 | 56.06 |

On day four, cultures are analyzed to determine the proliferation inhibition effects of zoledronic acid.

### EXAMPLE 7

### Drug Elusion Profiles of Zoledronic Acid from Coated Stents

Vascular stents such as, but not limited to, Medtronic Vascular S670, S660 and S7 are provided with polymer coatings containing zoledronic acid and the elusion profiles determined.

In vitro Drug Elution Studies

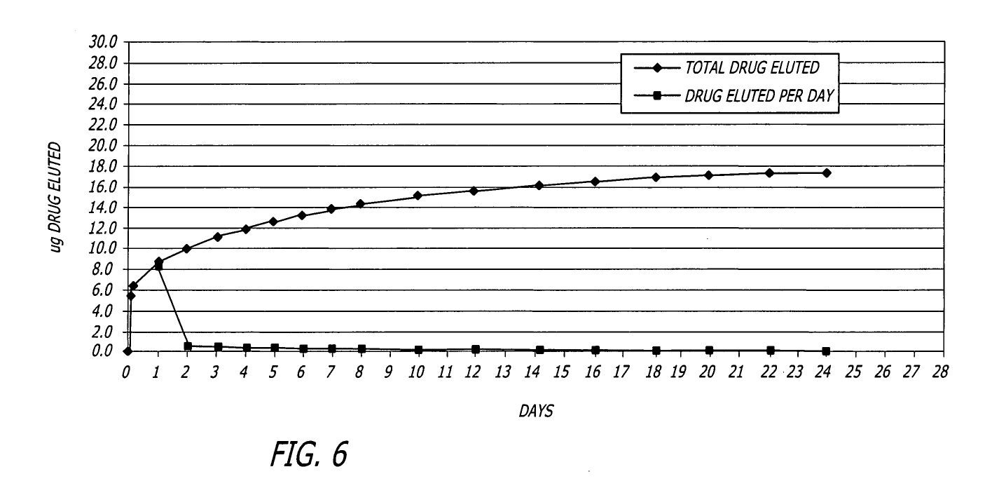

Fast zoledronic acid elutinq coating: An 18.0 mm long ×3.0 mm diameter stent is provided with a drug eluting polymer coating as described above. In this example the coating comprised a 75:25 polybutylmethacrylate-polyethylene vinyl acetate polymer blend containing 10% zoledronic acid by weight. The coated stents are incubated in 2 mL of elution media (0.4% SDS in 10 mM Tris, pH 6) that is pre-warmed to 37° C. The elution media is collected daily and replaced with 2 mL of pre-warmed elution media. The drug content is analyzed by HPLC using a water:acetonitrile gradient on a Waters NovaPack C18 column with detectection by UV at 304 nm wavelength. The elution profile depicted in FIG. 6 is a “fast elution” rate.

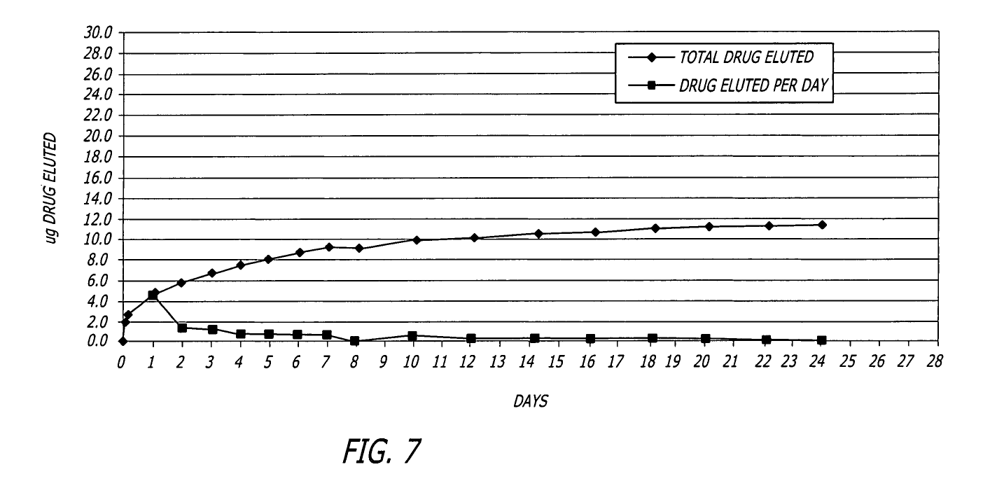

Slow zoledronic acid eluting coating: In another in vitro drug elution experiment an 18.0 mm long×3.0 mm diameter stent is provided with a drug eluting polymer coating comprised of an 80:20 polybutylmethacrylate-polyethylene vinyl acetate polymer blend containing 10% zoledronic acid by weight. The coated stents are incubated in 2 mL of elution media (0.4% SDS in 10 mM Tris, pH 6) that is pre-warmed to 37° C. The elution media is collected daily and replaced with 2 mL of pre-warmed elution media. The drug content is analyzed by HPLC using a water:acetonitrile gradient on a Waters NovaPack C18 column with detection by UV at 304 nm wavelength. The elution profile depicted in FIG. 7 is a “slow elution” rate.

In vivo Drug Elution Studies

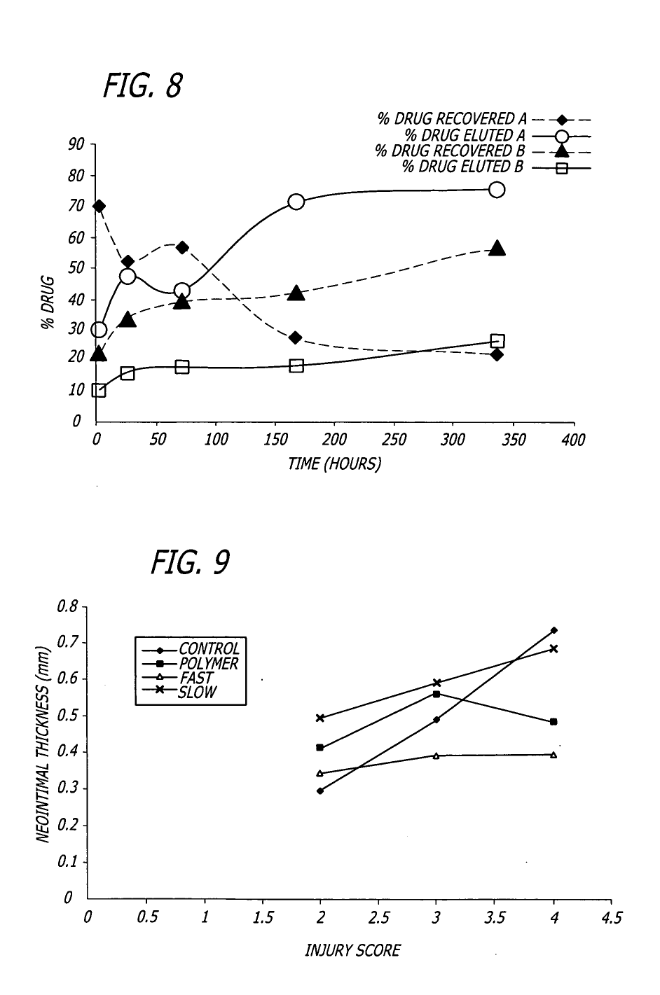

For in vivo studies stents having both fast and slow zoledronic acid eluting coatings are prepare as described above. The coated stents are implanted into rabbit iliac veins for a total of 336 hrs. At each time point depicted in FIG. 8 rabbits are euthanized and the stented vessels removed and reserved. After all stents are recovered from all time points the tissue around each stent is carefully removed, and the stents are incubated at 37° C. in dimethylsulfoxide (DMSO) until the remaining coating is stripped from the stent surface. The drug content of the DMSO is analyzed using HPLC as described above. The concentration of the drug remaining in the coating after removal from the rabbit iliac is inversely proportional to the total amount of drug eluted in vivo for a given time point. For comparison purposes, stents prepared identically to those used in vivo are incubated in vitro in elution buffer as described above and tested in parallel with the in vivo stents at each time point.

FIG. 8 graphically compares in vivo drug elution profiles with their corresponding in vitro drug elution profiles. In vivo drug elution profiles are depicted in dashed lines; in vitro drug elution profiles are depicted in solid lines. Stents having the “slow elution rate” coatings are represented by triangles for in vivo studies and open boxes for in vitro tests. “Fast elution rate” coatings are represented by diamonds for in vivo studies and open circles for in vitro tests.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it are individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A medical device for delivering an anti-restenotic composition comprising:
a stent having a generally cylindrical shape comprising an outer surface, an inner surface, a first open end, a second open end and wherein at least one of said inner or said outer surfaces are adapted to deliver an anti-restenotic effective amount of at least one bisphosphonate to a tissue within a mammal.

2. The medical device according to claim 1 wherein said stent is mechanically expandable.

3. The medical device according to claim 1 wherein said stent is self expandable.

4. The medical device according to claim 1 wherein said at least one bisphosphonate is present on both said inner surface and said outer surface of said stent.

5. The medical device according to claim 1 wherein at least one of said inner or said outer surfaces are coated with a polymer wherein said polymer has at least one bisphosphonate incorporated therein and said polymer releases said at least one bisphosphonate into said tissue of said mammal.

6. The medical device according to claim 1 wherein said at least one bisphosphonate inhibits or interferes with the normal biological function.

7. The medical device according to claim 6 wherein said bisphosphonate zolendronate or pamedronate.

8. The medical device according to claim 1 wherein said stent is delivered to said tissue using a balloon catheter.

9. The medical device according to claim 8 where said tissue is an anatomical lumen.

10. The medical device according to claim 9 wherein said anatomical lumen is a blood vessel lumen.

11. The medical device according to claim 5 wherein said polymer is selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose and combinations thereof.

12. A vascular stent comprising a polymeric coating containing an anti-restenotic effective amount of a bisphosphonate.

13. The vascular stent of claim 12 further comprising a parylene primer coat.

14. The vascular stent of claim 12 wherein said polymeric coating comprises a polybutylmethacrylate-polyethylene vinyl acetate polymer blend.

15. The vascular stent of claim 1 or claim 11 wherein said bisphosphonate is in a concentration of between 0.1% to 99% by weight of bisphosphonate-to-polymer.

16. The vascular stent according to claim 15 wherein said at least one bisphosphonate is zolendronate or pamidronic acid.

17. The vascular stent according to claim 16 wherein said stent is delivered to a tissue of a mammal's anatomical lumen using a balloon catheter.

18. A method for inhibiting or treating restenosis in a mammal comprising the site specific delivery of at least one bisphosphonate.

19. The method according to claim 18 wherein said bisphosphonate is delivered to a site at risk for restenosis using a vascular stent.

20. The method according to claim 18 wherein said bisphosphonate is delivered to a site at risk for restenosis using an injection catheter.

21. The method according to claim 18 wherein said bisphosphonate zolendronate or pamedronate.

22. A method for inhibiting or treating restenosis comprising providing a vascular stent having a coating comprising an anti-restenotic effective amount of zoledronic acid.

23. A method of inhibiting or treating restenosis in a mammal comprising the site specific delivery of at least one biphosphonate.
